SEQUENCE LISTING (4 sequences)

SEQ ID NO: 1 - protein, 10 aa, Andrias davidianus
TGPLGPSPGP

SEQ ID NO: 2 - protein, 9 aa, Andrias davidianus
HDLPLPLPE

SEQ ID NO: 3 - protein, 8 aa, Andrias davidianus
DNLPPLPK

SEQ ID NO: 4 - protein, 12 aa, Andrias davidianus
VPSGPLGPEGPR

(19) United States
(12) Patent Application Publication
(10) Pub. No.: US 2021/0047366 A1
(43) Pub. Date: Feb. 18, 2021

(54) ANDRIAS DAVIDIANUS CARTILAGE PREPARATION

(71) Applicant: TSINGHUA UNIVERSITY, Beijing (CN)

(72) Inventors: Xinhui XING, Beijing (CN); Yi WANG, Beijing (CN); Chong ZHANG, Beijing (CN); Nan SU, Beijing (CN)

(21) Appl. No.: 16/639,570

(22) PCT Filed: Jul. 5, 2018
(86) PCT No.: PCT/CN2018/094613

§ 371 Date: Aug. 21, 2020

(30) Foreign Application Priority Data
Aug. 15, 2017 (CN) 201710696668.8

(51) Int. Cl. C07K 1/30; C07K 7/08; C07K 7/06

(52) U.S. Cl. CPC C07K 1/30; C07K 7/06; C07K 7/08

(57) ABSTRACT

Disclosed is an Andrias davidianus cartilage preparation. The Andrias davidianus cartilage enzymatic extract of the present invention is obtained by a method comprising: obtaining cartilage from Andrias davidianus, proteolyzing the obtained cartilage, obtaining a supernatant after the proteolysis, and drying the supernatant. The Andrias davidianus cartilage enzymatic extract and an alcohol-soluble component thereof according to the present invention comprise a cartilage polypeptide, and have the effects of lowering uric acid, or treating hyperuricemia.

### TECHNICAL FIELD

The invention relates to an Andrias davidianus cartilage preparation and use thereof.

### BACKGROUND ART

Cartilage is rich in glycoproteins, peptides, small-molecule proteins, and polysaccharides. Studies have found that cartilage extracts from certain animals have good anti-tumor, anti-angiogenesis, immune regulation, and anti-arthritis activities. Among them, researches on shark cartilage preparation (SCP) are particularly abundant, and SCP is widely popular as a nutritional supplement worldwide. However, with the development of the shark cartilage market, the amount of shark fishing has increased. In recent decades, the number of shark populations in nature has dropped sharply, and some types of sharks have been included in the endangered species list. The use of shark cartilage is therefore unsustainable.

Currently, the cartilage preparations on the market are mainly derived from shark cartilage. With the development of the shark cartilage market, the amount of shark fishing has increased. In recent decades, the number of shark populations in nature has dropped drastically, and some types of sharks have been included in the endangered species list. The use of shark cartilage is therefore unsustainable.

### CONTENTS OF THE INVENTION

As a rare animal unique to China, giant salamander (Andrias davidianus) has high edible and medicinal value, and has great potential for development and utilization. Wild Andrias davidianus is a second-class protected aquatic animal in China, but in the past two decades, the artificial domestication and breeding technology for Andrias davidianus has gradually matured, and the Andrias davidianus industry has developed rapidly. Large-scale Andrias davidianus farming industries and the markets have been formed in many provinces and regions such as Shaanxi, Hunan, Henan, and Sichuan, and this provides sustainable biological resources for generating extract products with medicinal and health care value from Andrias davidianus cartilage, and the development of Andrias davidianus cartilage preparations is also conducive to the development of Andrias davidianus industry towards higher value.

Specifically, the present invention relates to the following items:

1. An Andrias davidianus cartilage enzymatic extract, comprising:

an Andrias davidianus chondroitin sulfate, and

a Crude cartilage peptide extract.

2. The Andrias davidianus cartilage enzymatic extract according to item 1, wherein the Andrias davidianus chondroitin sulfate contains a non-sulfated disaccharide (CS—O), a disaccharide sulfated at the C6 position (CS—C), and a disaccharide sulfated at the C4 position (CS-A).

3. The Andrias davidianus cartilage enzymatic extract according to item 1 or 2, wherein the Andrias davidianus chondroitin sulfate is composed of a non-sulfated disaccharide (CS—O), a disaccharide sulfated at the C6 position (CS—C), and a disaccharide sulfated at the C4 position (CS-A).

4. The Andrias davidianus cartilage enzymatic extract according to any one of items 1 to 3, wherein the Andrias davidianus chondroitin sulfate has a weight-average molecular weight (Mw) of 40 kDa or more, preferably 45 kDa or more, more preferably 48 kDa or more, most preferably 49.2 kDa.

5. The Andrias davidianus cartilage enzymatic extract according to any one of items 1 to 4, wherein the crude cartilage polypeptide extract comprises one or more of TGPLGPSPGP, HDLPLPLPE, DNLPPLPK, and VPSGPLGPEGPR.

6. The Andrias davidianus cartilage enzymatic extract according to any one of items 1 to 5, wherein the Andrias davidianus chondroitin sulfate accounts for 15% to 30% by mass of the total Andrias davidianus cartilage enzymatic extract, and the crude cartilage polypeptide extract accounts for 70% to 85% by mass of the total Andrias davidianus cartilage enzymatic extract.

7. The Andrias davidianus cartilage enzymatic extract according to any one of items 1 to 6, which is obtained by a method comprising:

obtaining cartilage from Andrias davidianus,

proteolyzing the obtained cartilage, and

obtaining a proteolyzed supernatant and drying the same.

8. An alcohol-soluble component of the Andrias davidianus cartilage enzymatic extract, which is a crude cartilage polypeptide extract, and comprises one or more of TGPLGPSPGP, HDLPLPLPE, DNLPPLPK, and VPSGPLGPEGPR.

9. The alcohol-soluble component according to item 8, which is obtained by a method comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a supernatant.

10. The alcohol-soluble component according to item 8 or 9, which has a uric acid-lowering activity.

11. A cartilage polypeptide, characterized in that the cartilage polypeptide comprises one or more of TGPLGPSPGP, HDLPLPLPE, DNLPPLPK, and VPSGPLGPEGPR.

12. The cartilage polypeptide according to item 11, which has an activity of inhibiting XOD enzyme.

13. The cartilage polypeptide according to item 11, which has uric acid-lowering activity.

14. An alcohol precipitation component of the Andrias davidianus cartilage enzymatic extract, comprising an Andrias davidianus chondroitin sulfate, wherein the Andrias davidianus chondroitin sulfate contains a non-sulfated disaccharide (CS—O), a disaccharide sulfated at the C6 position (CS—C), and a disaccharide sulfated at the C4 position (CS-A).

15. The alcohol precipitation component of the Andrias davidianus cartilage enzymatic extract according to item 14, wherein the Andrias davidianus chondroitin sulfate is composed of a non-sulfated disaccharide (CS—O), a disaccharide sulfated at the C6 position (CS—C), and a disaccharide sulfated at the C4 position (CS-A).

16. The alcohol precipitation component of the Andrias davidianus cartilage enzymatic extract according to item 14 or 15, wherein the Andrias davidianus chondroitin sulfate has a weight-average molecular weight (Mw) of 40 kDa or more, preferably 45 kDa or more, more preferably 48 kDa or more, most preferably 49.2 kDa.

17. The alcohol precipitation component of the Andrias davidianus cartilage enzymatic extract according to any one of items 14 to 16, which is obtained by a method comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a precipitate.

18. Use of the Andrias davidianus cartilage enzymatic extract according to any one of items 1 to 7, the alcohol-soluble component of the Andrias davidianus cartilage enzymatic extract according to any one of items 8 to 10, or the cartilage polypeptide according to any one of items 11 to 13 in the preparation of a medicament for reducing uric acid or treating hyperuricemia or gout.

19. Use of the Andrias davidianus cartilage enzymatic extract according to any one of items 1 to 7, the alcohol-soluble component of the Andrias davidianus cartilage enzymatic extract according to any one of items 8 to 10, or the cartilage polypeptide according to any one of items 11 to 13 in the reduction of uric acid or the treatment of hyperuricemia or gout.

20. A method for obtaining the Andrias davidianus cartilage enzymatic extract, comprising:

obtaining cartilage from Andrias davidianus,

proteolyzing the obtained cartilage, and

obtaining a proteolyzed supernatant and drying the same.

21. The method according to item 20, wherein the protease is an alkaline protease, and the proteolysis is performed under the conditions of pH 9-11.

22. A method for obtaining the alcohol-soluble component of the Andrias davidianus cartilage enzymatic extract, comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a supernatant.

23. The method according to item 22, wherein the protease is an alkaline protease, and the proteolysis is performed under the conditions of pH 9-11.

24. A method for obtaining the alcohol precipitation component of the Andrias davidianus cartilage enzymatic extract, comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a precipitate.

25. The method according to item 24, wherein the protease is an alkaline protease, and the proteolysis is performed under the conditions of pH 9-11.

26. The method according to any one of items 21, 23 or 25, wherein the alkaline protease is a 2709 protease.

27. The method according to any one of items 22 to 26, wherein the mass concentration of ethanol in the alcohol precipitation operation is 50%-90%.

28. The method according to item 27, wherein the mass concentration of ethanol in the alcohol precipitation operation is 70%.

29. A method for reducing uric acid or treating hyperuricemia or gout, comprising: administering the Andrias davidianus cartilage enzymatic extract according to any one of items 1 to 7, the alcohol-soluble component of the Andrias davidianus cartilage enzymatic extract according to any one of items 8 to 10, or the cartilage polypeptide according to any one of items 11 to 13 to the subject in need thereof.

The Andrias davidianus cartilage preparation according to the present invention is a polypeptide substance or a composition of Andrias davidianus chondroitin sulfate and a polypeptide, which is obtained by hydrolyzing the Andrias davidianus cartilage by a protease, and has anti-tumor, anti-angiogenesis, anti-inflammatory, anti-oxidation, immuno-enhancement, anti-osteoporosis, anti-rheumatic, anti-uric acid, anti-gout and other activities.

### SPECIFIC EMBODIMENTS

The present invention relates to an Andrias davidianus cartilage enzymatic extract, comprising: an Andrias davidianus chondroitin sulfate and a crude cartilage polypeptide extract.

The Andrias davidianus chondroitin sulfate accounts for 15% to 30% by mass, preferably 18% to 26% by mass of the total Andrias davidianus cartilage enzymatic extract, and the crude cartilage polypeptide extract accounts for 70% to 85% by mass, preferably 74% to 82% by mass of the total Andrias davidianus cartilage enzymatic extract.

Wherein the Andrias davidianus chondroitin sulfate contains a non-sulfated disaccharide (CS—O), a disaccharide sulfated at the C6 position (CS—C), and a disaccharide sulfated at the C4 position (CS-A); preferably, it is composed of a non-sulfated disaccharide (CS—O), a disaccharide sulfated at the C6 position (CS—C), and a disaccharide sulfated at the C4 position (CS-A).

The preparation process of the Andrias davidianus cartilage preparation according to the present invention is as follows:

Obtaining cartilage from Andrias davidianus.

Proteolysis: using one or more proteases, for example, a collagenase, an alkaline protease, a streptase, a trypsin, or a papain, etc., preferably an alkaline protease, to hydrolyze the Andrias davidianus cartilage under suitable conditions, and heating up after a period of time to inactivate the protease.

Isolation: allowing the solution to stand for precipitation (or centrifugation, filtration, etc.) after the reaction to obtain a supernatant.

Drying: naturally drying (or freeze-drying, rotary evaporating, etc.) to remove the water in the supernatant to obtain the desired composition of the Andrias davidianus chondroitin sulfate and the cartilage polypeptide, also referred to as GSCP in the present invention.

Ethanol or ethanol solution is added to the supernatant obtained in the separation step or the composition obtained in the drying step for alcohol precipitation (the ethanol content in the final solution is 50%-90%), the supernatant is obtained and dried to get the required alcohol-soluble component, which mainly consists of polypeptides, and is also referred to as GSCP2 in the present invention.

After further analysis, it is found that the alcohol-soluble component comprises TGPLGPSPGP, HDLPLPLPE, DNLPPLPK, and VPSGPLGPEGPR with uric acid-lowering activity. The alcohol precipitation component mainly consists of Andrias davidianus chondroitin sulfate (ADCS).

The present invention relates to an Andrias davidianus cartilage enzymatic extract, which is characterized in that it is obtained by a method comprising:

obtaining cartilage from Andrias davidianus,

proteolyzing the obtained cartilage, and

obtaining a proteolyzed supernatant and drying the same.

The present invention also relates to an Andrias davidianus cartilage enzymatic extract, which is characterized in that it comprises:

an Andrias davidianus chondroitin sulfate, and

a crude cartilage peptide extract.

The protease of the present invention is preferably an alkaline protease, and more preferably a 2709 protease.

The present invention also relates to an Andrias davidianus cartilage enzymatic extract, which is characterized in that the Andrias davidianus chondroitin sulfate has a weight-average molecular weight (Mw) of 49.2 kDa.

The present invention also relates to an Andrias davidianus cartilage enzymatic extract, which is characterized in that the crude cartilage polypeptide extract comprises one or more of TGPLGPSPGP, HDLPLPLPE, DNLPPLPK, and VPSGPLGPEGPR.

The present invention relates to a method for preparing the Andrias davidianus cartilage enzymatic extract, which is characterized in that the method comprises:

obtaining cartilage from Andrias davidianus,

proteolyzing the obtained cartilage, and

obtaining a proteolyzed supernatant and drying the same.

The present invention relates to an alcohol-soluble component of the Andrias davidianus cartilage enzymatic extract, which is characterized in that it is obtained by a method comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a supernatant.

The present invention also relates to an alcohol-soluble component of the Andrias davidianus cartilage enzymatic extract, which is characterized in that it is a crude cartilage polypeptide extract.

The present invention also relates to an alcohol-soluble component of the Andrias davidianus cartilage enzymatic extract, which is characterized in that it contains one or more of TGPLGPSPGP, HDLPLPLPE, DNLPPLPK, and VPSGPLGPEGPR.

The present invention relates to a method for preparing the alcohol-soluble component of the Andrias davidianus cartilage enzymatic extract, which is characterized in that the method comprises:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a supernatant.

The present invention relates to a cartilage polypeptide, which is characterized in that it comprises one or more of TGPLGPSPGP, HDLPLPLPE, DNLPPLPK, and VPSGPLGPEGPR.

That is, the cartilage polypeptide of the present invention may be TGPLGPSPGP, HDLPLPLPE, DNLPPLPK, or VPSGPLGPEGPR, and any mixture thereof, for example, a mixture of TGPLGPSPGP and HDLPLPLPE; a mixture of TGPLGPSPGP and DNLPPLPK; a mixture of TGPLGPSPGP and VPSGPLGPEGPR; a mixture of HDLPLPLPE and DNLPPLPK; a mixture of HDLPLPLPE and VPSGPLGPEGPR; a mixture of DNLPPLPK and VPSGPLGPEGPR; a mixture of TGPLGPSPGP, HDLPLPLPE and DNLPPLPK; a mixture of HDLPLPLPE, DNLPPLPK and VPSGPLGPEGPR; a mixture of TGPLGPSPGP, HDLPLPLPPE and VPSGPLGPEGPR; a mixture of HDLPLPLPE, DNLPPLPK and VPSGPLGPEGPR; a mixture of TGPLGPSPGP, HDLPLPLPE, DNLPPLPK, and VPSGPLGPEGPR.

The present invention also relates to a cartilage polypeptide, which is characterized in that it has an activity of inhibiting XOD enzyme.

The present invention also relates to a cartilage polypeptide, which is characterized in that it has uric acid-lowering activity.

The present invention relates to an alcohol precipitation component of the Andrias davidianus cartilage enzymatic extract, which is characterized in that it is obtained by a method comprising:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a precipitate.

The present invention also relates to an alcohol precipitation component of the Andrias davidianus cartilage enzymatic extract, which is characterized in that it comprises an Andrias davidianus chondroitin sulfate.

The present invention also relates to an alcohol precipitation component of the Andrias davidianus cartilage enzymatic extract, which is characterized in that the Andrias davidianus chondroitin sulfate has a weight-average molecular weight (Mw) of 49.2 kDa.

The present invention relates to a method for preparing the alcohol precipitation component of the Andrias davidianus cartilage enzymatic extract, which is characterized in that the method comprises:

obtaining cartilage from Andrias davidianus,

proteolyzing the obtained cartilage,

obtaining a proteolyzed supernatant and drying the same,

alcohol-precipitating the proteolyzed supernatant or the dried matter thereof, and

obtaining a precipitate.

The Andrias davidianus cartilage enzymatic extract, or the alcohol-soluble component of the Andrias davidianus cartilage enzymatic extract, or the cartilage polypeptide, such as TGPLGPSPGP, HDLPLPLPE, DNLPPLPK, or VPSGPLGPEGPR according to the present invention has an activity of inhibiting XOD enzyme.

The present invention also relates to use of the Andrias davidianus cartilage enzymatic extract in the preparation of a medicament for reducing uric acid or treating hyperuricemia.

The present invention also relates to use of the Andrias davidianus cartilage enzymatic extract in the reduction of uric acid or in the treatment of hyperuricemia.

The present invention also relates to use of the Andrias davidianus cartilage enzymatic extract in the preparation of a medicament for treating gout.

The present invention also relates to use of the Andrias davidianus cartilage enzymatic extract in the treatment of gout.

The present invention also relates to use of the alcohol-soluble component of the Andrias davidianus cartilage enzymatic extract in the preparation of a medicament for reducing uric acid or treating hyperuricemia.

The present invention also relates to use of the alcohol-soluble component of the Andrias davidianus cartilage enzymatic extract in the reduction of uric acid or the treatment of hyperuricemia.

The present invention also relates to use of the alcohol-soluble component of the Andrias davidianus cartilage enzymatic extract in the preparation of a medicament for treating gout.

The present invention also relates to use of the alcohol-soluble component of the Andrias davidianus cartilage enzymatic extract in the treatment of gout.

The present invention also relates to use of the cartilage polypeptide such as TGPLGPSPGP, HDLPLPLPE, DNLPPLPK or VPSGPLGPEGPR in the preparation of a medicament for reducing uric acid or treating hyperuricemia.

The present invention also relates to use of the cartilage polypeptides such as TGPLGPSPGP, HDLPLPLPE, DNLPPLPK or VPSGPLGPEGPR in the reduction of uric acid or the treatment of hyperuricemia.

The present invention also relates to use of the cartilage polypeptide such as TGPLGPSPGP, HDLPLPLPE, DNLPPLPK or VPSGPLGPEGPR in the preparation of a medicament for treating gout.

The present invention also relates to use of the cartilage polypeptides such as TGPLGPSPGP, HDLPLPLPE, DNLPPLPK or VPSGPLGPEGPR in the treatment of gout.

Therefore, the Andrias davidianus cartilage enzymatic extract and the alcohol-soluble component thereof according to the present invention comprise a cartilage polypeptide, and have the effects of reducing uric acid, or treating hyperuricemia or gout.

### BRIEF DESCRIPTION OF THE DRAWINGS

FIG. 1 shows cellulose acetate membrane electrophoresis of GSCP components (HA: hyaluronic acid; CS: chondroitin sulfate; HP: heparin).

FIG. 2 shows HPGPC spectra of ADCS, shark chondroitin sulfate (SCS), and bovine chondroitin sulfate (BCS).

FIG. 3 shows the SAX-HPLC spectrum and disaccharide composition: (a) ADCS; (b) BCS; (c) SCS.

FIG. 4 shows the ¹H-NMR spectrum of ADCS.

FIG. 5 shows the total ion flow graph.

FIG. 6 shows the results of MALDI-TOF-MS analysis.

FIG. 7 shows the results of XOD enzyme activity detection.

FIG. 8 shows the evaluation of uric acid-lowering effect by a high-efficiency uricemia animal model: (a) blood uric acid content; (b) liver XOD enzyme activity.

### EXAMPLES

### Example 1: Preparing Andrias davidianus Cartilage Preparation

The segmented Andrias davidianus meat with cartilage is put into clean water, heating and boiling for 1 hour, and 20 g of Andrias davidianus cartilage is separated; the cartilage is placed into a 500 ml beaker, adding 200 ml of water and 2 g of 2709 protease (Beijing Donghua Qiangsheng Biological Company, derive from Bacillus licheniformis), hydrolyzing at 50° C., pH 9.5 for 8 h, then heating at 90° C. for 10 minutes to inactivate the protease; centrifuging at 10,000 rpm for 10 min and taking the supernatant; then the supernatant is vacuum freeze-dried for 48 h to obtain 10.6 g of solid powder, that is, to obtain the giant salamander cartilage enzymatic preparation (i.e., GSCP). 5 g of Andrias davidianus cartilage enzymatic extract is taken to mix well with 20 ml of 75% ethanol, centrifuging at 3,000 rpm for 5 minutes and taking the supernatant to place in an oven at 60° C. for 6 hours to remove alcohol, and then freeze-drying for 48 hours to obtain 3.5 g of alcohol-soluble component (i.e., GSCP2). The precipitate fraction is dried to obtain 1.2 g of Andrias davidianus chondroitin sulfate (i.e., ADCS). The alcohol precipitation fraction accounts for 25.5% by mass, and the alcohol-soluble fraction accounts for 74.5% by mass.

### Example 2: Preparing Andrias davidianus Cartilage Preparation

The Andrias davidianus (3.2 kg) used to prepare cartilage is isolated, after cleaning and washing, the Andrias davidianus cartilage with a wet weight of 100 g is obtained. The cartilage is cut into small pieces and homogenized with a homogenizer. The homogenized cartilage is suspended in 500 mL of a solution containing 2% sodium acetate, adjusting the pH to the range of 10.0±0.5 with 1M NaOH, adding 6 g of 2709 alkaline protease and heating with stirring, then adjusting the pH to the range of 10.0±0.5 with 1M NaOH every 30 min; the reaction is performed at 50° C. for about 6 hours until the cartilage tissue is completely digested, and then the reaction solution is heated to 100° C. for 10 minutes to inactivate the protease. After cooling to room temperature, the reaction solution is centrifuged at 5,000×g for 15 minutes to obtain the supernatant, and a solution of giant salamander cartilage enzymatic preparation (GSCP) is obtained. The supernatant is taken to add in 3 times the volume of absolute ethanol, leaving it at 4° C. overnight, centrifuging at 5,000×g for 15 min to collect a precipitate, dissolving the precipitate in 100 mL of deionized water and repeating the alcohol precipitation operation again to obtain a precipitate, and dissolving the precipitate in 100 mL of deionized water and dialyzing for 24 h by using a dialysis bag with a cut-off of 7 kDa, then freeze drying is performed to obtain 4.4 g of purified Andrias davidianus chondroitin sulfate. Supernatants from two alcohol precipitation operations are collected to concentrate by rotary evaporation, and then freeze-drying to obtain 19.0 g of an alcohol-soluble component mainly consisting of Andrias davidianus cartilage polypeptide. The alcohol precipitation fraction accounts for 18.8% by mass, and the alcohol-soluble fraction accounts for 81.2% by mass.

### Example 3: Analyzing Glycosaminoglycan Composition of GSCP Component Obtained in Example 1 by Cellulose Acetate Membrane Electrophoresis

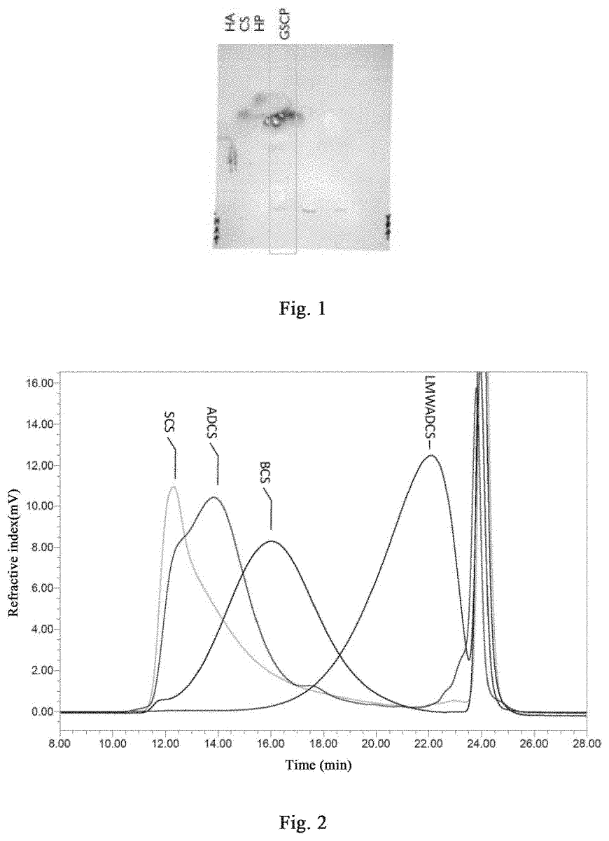

Cellulose acetate membrane (7 cm*9 cm) is thoroughly soaked in electrode buffer solution (0.1 mol/L pyridine, 0.47 mol/L formic acid, pH 3.0) for 30 minutes, then taking it out to remove and absorb the excess buffer solution. Samples are applied at one end of the cellulose acetate film by using a sampler (10 mg/ml GSCP), taking 2 mg/ml hyaluronic acid, chondroitin sulfate, and heparin samples as controls; running electrophoresis on a DYCP-38C horizontal electrophoresis instrument at a constant current of 6 mA for 30 minutes, staining in a staining solution (0.5% alcian blue, 2% acetic acid) for 30 minutes, and rinsing repeatedly with rinsing solution (2% acetic acid) 3-5 times for 20 minutes each time, then the rinsed cellulose acetate membrane is observed under visible light, and the results are shown in FIG. 1. FIG. 1 shows that the GSCP sample has a higher content of chondroitin sulfate.

### Example 4: Characterization of Andrias davidianus Chondroitin Sulfate

As mentioned earlier, cartilage with a wet weight of 100 g is isolated from a farmed Andrias davidianus with a wet weight of 3.2 kg. According to its wet-to-dry ratio (W/D, 5.34), the dry weight of cartilage for extracting CS (chondroitin sulfate) is 18.7 g. After extraction, separation, purification and freeze-drying, 4.4 g of ADCS sample is obtained in the experiment. The purity, molecular weight distribution, disaccharide composition, and charge density of ADCS and commercially available BCS and SCS are analyzed by HPGPC and SAX-HPLC. The results are shown in Table 1. The purity of ADCS is 98.7%, and the yield of ADCS extracted from the Andrias davidianus cartilage is 23.2% (dry weight/dry weight).

TABLE 1
Chemical properties of CS (purity, molecular weight distribution,
disaccharide composition, and charge density)

| CS | purity | Mn | Mw | dispersion | Disaccharide composition | | | | Charge |
|---|---|---|---|---|---|---|---|---|---|
| sample | (%) | (kDa) | (kDa) | coefficient (P) | CS-O | CS-C | CS-A | CS-D | density |
| ADCS | 98.7 | 43.5 | 49.2 | 1.13 | 0.15 | 0.61 | 0.24 | — | 0.85 |
| BCS | 99.0 | 30.6 | 34.4 | 1.13 | 0.05 | 0.36 | 0.59 | — | 0.95 |
| SCS | 98.2 | 51.9 | 75.8 | 1.46 | 0.02 | 0.49 | 0.30 | 0.19 | 1.17 |

The molecular weight distribution of CS is analyzed by HPGPC. The chromatogram is shown in FIG. 2. The dispersion coefficients of the three CS samples are less than 1.5, and the molecular weight distribution is relatively concentrated. The weight-average molecular weight (Mw) of ADCS is 49.2 kDa, which is lower than 75.8 kDa of SCS derived from aquatic animal sharks, and higher than 34.4 kDa of BCS derived from land animal bovine.

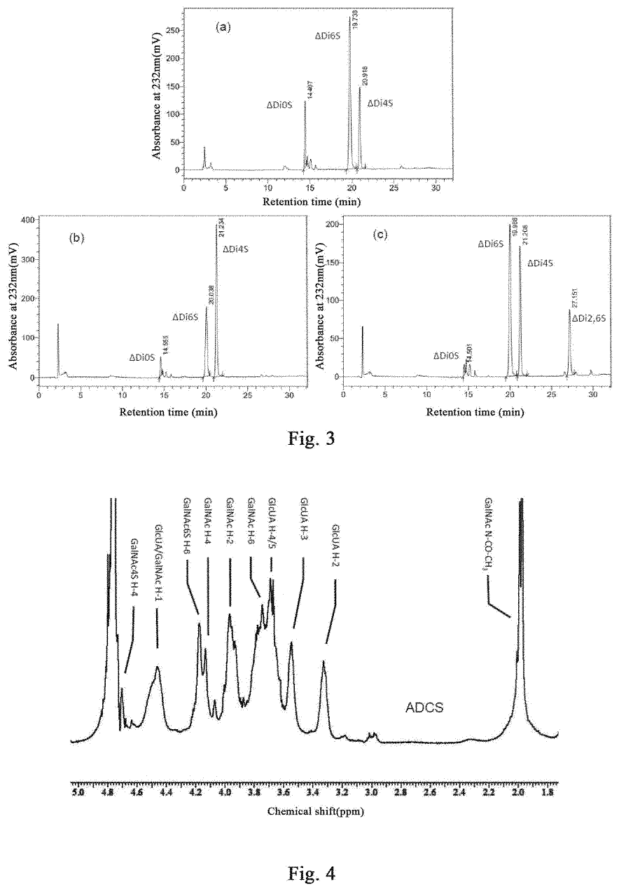

After being thoroughly degraded by the ChonABC enzyme, the disaccharide composition of CS is analyzed by SAX-HPLC, and the results are shown in FIG. 3. ADCS contains 14.6% of a non-sulfated disaccharide (CS—O), 60.9% of a disaccharide sulfated at the C6 position (CS—C), and 24.5% of a disaccharide sulfated at the C4 position (CS-A); and it does not contain disulfated disaccharide (CS-D). At the same time, it can be seen from Table 1 that, the disaccharide composition of ADCS is significantly different from that of BCS and SCS. Since the shark-derived CS (SCS) has high content of CS—C, it is often called CSC, while our experimental results show that the CS—C content in ADCS is as high as 60% or more, which is higher than that of the shark-derived CS. At the same time, the content of the non-sulfated disaccharide CS—O in ADCS is higher than that in BCS and SCS; as compared with SCS, ADCS does not contain the disulfated disaccharide units, so the charge density of ADCS (0.85) is also lower than that of BCS (0.95) and SCS (1.17). In addition, CS also extracted from amphibian cartilage, the disaccharide composition of ADCS is also significantly different from the frog-derived CS (40% CS—O, 40% CS-A, 20% CS—C), which is reported in the literature (Mathews M B, Hinds L D. Acid mucopolysaccharides of frog cartilage in thyroxine-induced metamorphosis[J]. Biochimica Et Biophysica Acta, 1963, 74(2): 198).

The ¹H-NMR spectrum analysis results of ADCS are shown in FIG. 4. Analysis of NMR spectrum is performed in conjunction with literature reports on CS nuclear magnetic resonance analysis (Mucci A, Schenetti L, Volpi N. H and 13 C nuclear magnetic resonance identification and characterization of components of chondroitin sulfates of various origin[J]. 2000, 41: 37-45). A group of overlapping peaks at 1.9-2.1 ppm belong to the signal peaks of the H atom in the methyl group of the unsulfated and sulfated GaINAc structure; the signal peaks at 4.18 ppm and 4.70 ppm respectively belong to the H atom at the position C6 of GalNAc6S in CS—C and the H atom at the position C4 of GalNAc4S in CS-A; the signal peak at 4.46 ppm belongs to the H atom at the position C1 of GlcUA and GaINAc; the signal peaks at 3.32 ppm, 3.54 ppm, and 3.69 ppm respectively belong to the H atom at the positions C2, C3 and C4/C5 of GlcUA.

It can be seen that, after analysis, it is found that. ADCS with a purity of 98% or more is obtained by alcohol precipitation operation and dialysis purification of the Andrias davidianus cartilage enzymatic extract. The molecular weight of ADCS is determined to be 49.2 kDa by HPGPC analysis. After being completely degraded by the ChonABC enzyme, the ADCS disaccharide composition is analyzed by SAX-HPLC, and it consist of 14.6% CS—O, 60.9% CS—C, and 24.5% CS-A, therefore, the ADCS disaccharide composition is significant distinct from that of the BCS from cattle and the SCS from shark.

### Example 5: Amino Acid Composition, Polypeptide Molecular Weight Distribution, and Protein Sequencing of the Andrias davidianus Cartilage Enzymatic Extract (GSCP) and the Alcohol Soluble Component Thereof (GPC2)

Analysis 1: The amino acid composition analysis of GSCP was entrusted to Beijing Agricultural Biological Testing Center.

I. Sample Information

Sample type: biological sample

Storage condition: −20° C.

Number of samples: 7

Test content: Detection of 50 kinds of hydrolyzed amino acids

2. Instrument and Reagent Information

Technology used: mass spectrometry-isotope internal standard

Instrument: high performance liquid chromatography-quadrupole ion trap tandem mass spectrometer (HPLC-MS/MS Q-TRAP)

Instrument Model: HPLC-MS/MS (Ultimate3000-API 3200 Q TRAP)

Amino acid kit: API 45AA kit; methanol and nitrile were purchased from fisher.

3. Sample Pre-Treatment

Acid hydrolysis treatment (solid sample: soaking-homogenization-centrifugation)

(1) Taking 100 ul sample, adding 400 ul water, adding 500 ul concentrated hydrochloric acid, mixing and filling with nitrogen for protection, sealing at 110° C., and then digesting at high temperature for 21 hours.

(2) Taking out the digested solution, centrifuging and taking the supernatant, drying 50 ul of the supernatant by nitrogen-blowing, and reconstituting with 1 ml of water.

Derivatization

(1) Taking 40 μL of the complex solution to put it into a test tube, adding 10 μL of sulfosalicylic acid, and vortexing for 30 seconds, then centrifuging at 13200 rpm for 4 minutes.

(2) Taking 10 μL of the upper liquid to put it into another test tube, adding 40 μL. of labeling buffer, and vortexing to mix well, then performing rotary centrifugation.

(3) Taking 10 μL of the upper layer liquid to put it into another test tube, adding 5 μL of diluted aTRAQ reagent to each sample tube, and vortexing to mix well, then performing rotary centrifugation.

(4) Incubating at room temperature for at least 30 minutes, adding 5 μL of hydroxylamine into the tube, and vortexing to mix well, then performing rotary centrifugation.

(5) Adding 32 μL of internal standard to each tube, vortexing to mix well, and performing rotary centrifugation, then preserving for inspection.

4. The Experimental Parameters

(1) Liquid phase conditions:

Column: MSLab HP-C18 (150*4.6 mm 5 um)

Mobile phase: A: water+0.1% formic acid B: acetonitrile+0.1% formic acid

Flow rate: 0.8 ml/min

Column temperature: 50° C.

Injection volume: 3 ul

Liquid phase analysis gradient:

| Time (min) | Flow rate | A (%) | B (%) |
|---|---|---|---|
| 0 | 0.8 | 98 | 2 |
| 10 | 0.8 | 72 | 28 |
| 10.1 | 0.8 | 0 | 100 |
| 16 | 0.8 | 0 | 100 |
| 16.1 | 0.8 | 98 | 2 |
| 25 | 0.8 | 98 | 2 |

(2) MS conditions:

Ion source: +ESI electrospray ion source, positive ion mode

Scanning method: MRM multiple reaction monitoring

CUR: 20 (curtain gas)

CAD: Medium (collision gas)

IS: +5500V (ionspray voltage)

TEM: 500° C. (atomization temperature)

GS1: 55 psi (atomizing gas)

GS2: 60 psi (auxiliary gas)

DP: 35 v (declustering potential)

EP: 10 (injection voltage)

CE: 30 (collision energy)

CXP: 5.0 (injection voltage of collision chamber)

5. Experimental Data

(1) Total ion current diagram

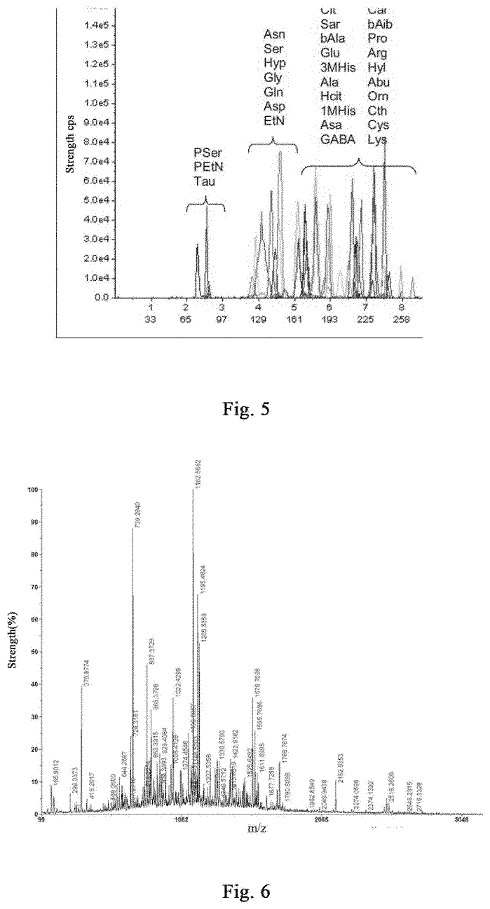

The total ion current diagram is shown in FIG. 5.

(2) Testing results, see Table 2.

TABLE 2
Amino acid composition test results

| | | | GSCP | cartilage | skin | mucus | muscle | muscle |
|---|---|---|---|---|---|---|---|---|
| Amino acid | | | Content | (dry) | (dry) | (dry) | (dry) | (wet) |
| number | abbreviation | English name | mg/ml | content % | | | | |
| AA01 | Ser | Serine | 1.6478 | 1.8332% | 2.3364% | 3.3856% | 1.7648% | 0.3000% |
| AA02 | Gly | Glycine | 19.5182 | 7.8406% | 14.6207% | 9.4998% | 2.7208% | 0.4625% |
| AA03 | His | Histidine | 0.1828 | 0.6163% | 0.4100% | 1.3036% | 0.7772% | 0.1321% |
| AA04 | Thr | Threonine | 1.5177 | 1.2442% | 1.0312% | 5.3731% | 2.2176% | 0.3770% |
| AA05 | Glu | Glutamic acid | 4.7376 | 3.8009% | 3.9883% | 4.3229% | 5.2766% | 0.8970% |
| AA06 | Gln | Glutamine | 0.0030 | 0.0069% | 0.0000% | 0.0000% | 0.0000% | 0.0000% |
| AA07 | Asp | Aspartic acid | 0.0000 | 0.0000% | 0.0000% | 0.0000% | 0.0000% | 0.0000% |
| AA08 | Asn | Asparagine | 0.0000 | 0.0000% | 0.0000% | 0.0000% | 0.0000% | 0.0000% |
| AA09 | Ala | Alanine | 0.1741 | 0.1223% | 0.1762% | 0.1470% | 0.1190% | 0.0202% |
| AA10 | Arg | Arginine | 2.0381 | 2.6807% | 4.1268% | 3.5693% | 3.7022% | 0.6294% |
| AA11 | Pro | Proline | 4.4214 | 3.2495% | 4.0391% | 2.9824% | 1.4879% | 0.2529% |
| AA12 | Cys | Cysteine | 0.0640 | 0.2040% | 0.0657% | 0.4668% | 0.4207% | 0.0715% |
| AA13 | Lys | Lysine | 2.8566 | 2.2010% | 2.2178% | 5.8212% | 5.1874% | 0.8819% |
| AA14 | Met | Methionine | 0.7370 | 0.4468% | 0.5472% | 0.9767% | 1.4552% | 0.2474% |
| AA15 | Val | Valine | 2.2751 | 1.2952% | 1.0274% | 3.9402% | 2.2674% | 0.3855% |
| AA16 | Tyr | Tyrosine | 1.0038 | 0.7761% | 0.4953% | 4.5998% | 1.9872% | 0.3378% |
| AA17 | Ile | Isoleucine | 1.5847 | 1.0713% | 0.6753% | 3.3978% | 2.0262% | 0.3445% |
| AA18 | Leu | Leucine | 2.6496 | 1.4866% | 1.3027% | 3.5666% | 3.3353% | 0.5670% |
| AA19 | Phe | Phenylalanine | 1.9525 | 1.1655% | 1.1590% | 4.0233% | 2.2412% | 0.3810% |
| AA20 | Trp | Tryptophan | 0.0000 | 0.0000% | 0.0000% | 0.0076% | 0.0000% | 0.0000% |
| AA21 | PSer | Phospho-serine | 0.0000 | 0.0000% | 0.0000% | 0.0000% | 0.0000% | 0.0000% |
| AA22 | PEtN | Phosphorylethanol | 0.0006 | 0.0000% | 0.0000% | 0.0000% | 0.0005% | 0.0001% |
| | | amine | | | | | | |
| AA23 | Tau | Taurine | 0.0077 | 0.0108% | 0.0057% | 0.0024% | 0.0034% | 0.0006% |
| AA24 | Hyp | Hydroxy-proline | 1.7335 | 0.8232% | 1.1231% | 0.0291% | 0.0131% | 0.0022% |
| AA25 | EtN | Ethanolamine | 0.0074 | 0.0092% | 0.0086% | 0.0631% | 0.0554% | 0.0094% |
| AA26 | Cit | Citrulline | 0.0484 | 0.0000% | 0.0000% | 0.0000% | 0.0000% | 0.0000% |
| AA27 | Hcit | Homocitrulline | 0.0000 | 0.0000% | 0.0008% | 0.0013% | 0.0000% | 0.0000% |
| AA28 | Orn | Ornithine | 0.8088 | 0.0000% | 0.0000% | 0.0000% | 0.0000% | 0.0000% |
| AA29 | Asa | argininosuccinic | 0.0000 | 0.0000% | 0.0000% | 0.0000% | 0.0000% | 0.0000% |
| | | acid | | | | | | |
| AA30 | Sar | Sarcosine | 0.0140 | 0.0000% | 0.0012% | 0.0000% | 0.0091% | 0.0015% |
| AA31 | bAla | b-Alanine | 0.1400 | 0.0098% | 0.0428% | 0.0212% | 0.3888% | 0.0661% |
| AA32 | Car | Carnosine | 0.0000 | 0.0000% | 0.0000% | 0.0000% | 0.0000% | 0.0000% |
| AA33 | Ans | Anserine | 0.0000 | 0.0000% | 0.0000% | 0.0000% | 0.0000% | 0.0000% |
| AA34 | Aad | a-Aminoadipic | 0.0121 | 0.0064% | 0.0000% | 0.0000% | 0.0002% | 0.0000% |
| | | acid | | | | | | |
| AA35 | GABA | r-Aminobutyric | 0.0107 | 0.0014% | 0.0004% | 0.0001% | 0.0020% | 0.0003% |
| | | acid | | | | | | |
| AA36 | 3Aib | 3Aminoisobutyric | 0.0038 | 0.0001% | 0.0001% | 0.0000% | 0.0009% | 0.0002% |
| | | acid | | | | | | |
| AA37 | 2Aib | 2Aminoisobutyric | 0.0027 | 0.0000% | 0.0000% | 0.0000% | 0.0000% | 0.0000% |
| | | acid | | | | | | |
| AA38 | Abu | 2Aminobutyric | 0.0681 | 0.0000% | 0.0004% | 0.0006% | 0.0015% | 0.0002% |
| | | acid | | | | | | |
| AA39 | Hyl | 5Hydroxylysine | 0.0000 | 0.0000% | 0.0000% | 0.0013% | 0.0000% | 0.0000% |
| AA40 | 5Ava | 5Aminovaleric | 0.0143 | 0.0005% | 0.0003% | 0.0005% | 0.0006% | 0.0001% |
| | | acid | | | | | | |
| AA41 | 6ACA | Aminocaproic | 0.0000 | 0.0000% | 0.0000% | 0.0000% | 0.0000% | 0.0000% |
| | | Acid | | | | | | |
| AA42 | Hcy | Homocysteine | 0.0000 | 0.0000% | 0.0000% | 0.0020% | 0.0000% | 0.0000% |
| AA43 | Cth | cystathionine | 0.0000 | 0.0000% | 0.0000% | 0.0000% | 0.0000% | 0.0000% |
| AA44 | 5HT | 5-Hydroxy- | 0.0000 | 0.0000% | 0.0000% | 0.0000% | 0.0000% | 0.0000% |
| | | Tryptamine | | | | | | |
| AA45 | 5HTP | 5-Hydroxy- | 0.0000 | 0.0000% | 0.0000% | 0.0000% | 0.0000% | 0.0000% |
| | | tryptophan | | | | | | |
| AA46 | Kyn | kynurenine | 0.0006 | 0.0009% | 0.0004% | 0.0055% | 0.0007% | 0.0001% |
| AA47 | 3MHis | 3Methyl- | 0.0028 | 0.0000% | 0.0029% | 0.0000% | 0.0343% | 0.0058% |
| | | histidine | | | | | | |
| AA48 | Harg | Homoarginine | 0.0000 | 0.0000% | 0.0000% | 0.0000% | 0.0000% | 0.0000% |
| AA49 | Hpro | Pipecolic acid | 0.1057 | 0.1313% | 0.0668% | 1.1634% | 0.8480% | 0.1442% |
| AA50 | Hser | Homoserine | 0.0034 | 0.0028% | 0.0016% | 0.0018% | 0.0019% | 0.0003% |
| | | | 50.35 | 31.04% | 39.47% | 58.68% | 38.3469% | 6.52% |

Note:
GSCP samples are 100 mg/ml solution, others are solid samples

Analysis 2: Peptide molecular weight distribution, protein sequencing of the alcohol-soluble fraction (GSCP2)

The alcohol-soluble fraction has significant uric acid-lowering activity, and it is further purified by gel size exclusion chromatography and reversed-phase preparative chromatography to obtain a component having significant inhibitory effect on XOD enzyme activity, the molecular weights and sequence analysis of the polypeptide are performed, the method and the results are as follows:

The molecular weight and sequence analysis of polypeptide samples were entrusted to the Tsinghua University Biomedical Testing Center, wherein the molecular weight analysis is performed by using a matrix-assisted laser analytical time-of-flight mass spectrometer MALDI-TOF-TOF 4800Plus (ABI, USA); the polypeptide sequence analysis is performed by using OrbiTrap Fusion LUMOS Liquid Chromatograph Mass Spectrometer (Thermo Fisher Company, USA).

The results of MALDI-TOF-MS analysis are shown in FIG. 6. The analysis results show that the mass-to-charge ratio (m/z) of the sample peptide is in the range of 100-3000, and both samples have strong peaks at m/z 739 and 1162.

The samples are analyzed by liquid chromatography-electrospray tandem mass spectrometry (LC-ESI-MS/MS), and the amino acid sequences of the polypeptides are analyzed by the data-independent acquisition (DIA) scheme, the resulting four major polypeptide sequences are shown in Table 3, they are respectively named as S1, S2, S3, and S4, and are artificially synthesized.

TABLE 3
Sequence analysis of the four major polypeptides

| | | | Relative |
|---|---|---|---|
| | Amino acid | Number of | molecular |
| Peptides | sequences | amino acids | weight |
| S1 | VPSGPLGPEGPR | 12 | 1161.614 |
| S2 | TGPLGPSPGP | 10 | 878.4498 |
| S3 | HDLPLPLPE | 9 | 1029.549 |
| S4 | DNLPPLPK | 8 | 892.5018 |

### Example 6: Uric Acid-Lowering Activity of the Enzymatic Extract (GSCP) and the Alcohol-Soluble Component (GSCP2)

Analysis 1:

The oxidation of xanthine is catalyzed by XOD (xanthine oxidase) to generate uric acid and superoxide anions, and it is one of the main sources of reactive oxygen species. XOD is also one of the key enzymes for nucleotide metabolism, and is mainly distributed in mammals' heart, lung, liver and other tissues. The XOD enzyme activity inhibition model may be used to screen and evaluate potential uric acid lowering drugs.

Reagents: XOD enzyme (Sigma, derived from milk), XOD enzyme activity detection kit (purchased from Nanjing Jiancheng Bioengineering Institute).

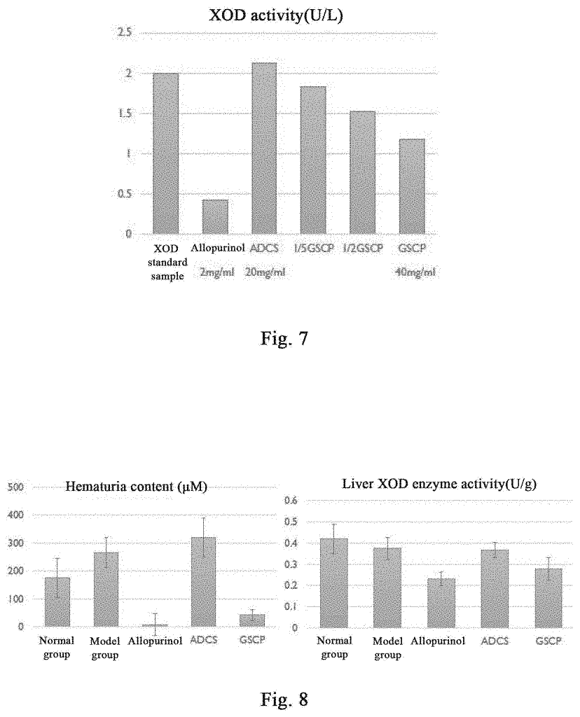

The oxidation of xanthine is catalyzed by Xanthine oxidase (XOD) to generate uric acid with specific absorption at 295 nm. The enzymatic activity may be characterized by spectrophotometric determination of the product. The XOD enzyme activity detection is performed according to the instructions of the XOD enzyme activity detection kit, and the results are shown in FIG. 7. It can be seen from the results in FIG. 7 that, GSCP has XOD-inhibiting activity and is dose-dependent. The inhibitory activity is the greatest when the concentration of GSCP is 40 mg/ml, after further diluting to ½ or ⅕ concentration the inhibitory activity decreases accordingly.

In addition, it can be seen from FIG. 7 that, ADCS, which is contained in a large amount in GSCP, does not have XOD inhibitory activity. It is suggested that the component having the activity of inhibiting XOD enzyme is a polypeptide component in GSCP, i.e., a component of GSCP2.

In the absence of inhibitors, the XOD enzyme activity is 100%. The XOD enzyme activity inhibitory effects of the synthetic uric acid-lowing active peptide and the allopurinol (positive drug) are compared. The results are shown in Table 4 (the concentrations in the table are the final concentrations of the samples in the reaction system).

TABLE 4
XOD inhibitory activity of GSCP2, the
synthetic polypeptides and allopurinol

| | 2.5 | 5 | 10 |
|---|---|---|---|
| GSCP2(mg/ml) | | | |
| XOD activity (%) | 64.6 | 50.9 | 43.3 |
| S1(mg/ml) | | | |
| XOD activity (%) | 57.6 | 36.7 | 26.9 |
| S2(μg/ml) | 50 | 100 | 200 |
| XOD activity (%) | 80.0 | 55.8 | 17.9 |
| | 20 | 40 | 80 |
| S3(μg/ml) | | | |
| XOD activity (%) | 87.2 | 67.3 | 46.9 |
| S4(μg/ml) | | | |
| XOD activity (%) | 87.9 | 67.7 | 43.2 |

| Allopurinol (μg/ml) | 0.2 | 0.4 | 0.8 |
|---|---|---|---|
| XOD activity (%) | 59.4 | 34.7 | 31.2 |

The XOD enzyme activity inhibition experiment is used to calculate the half-inhibitory concentrations (IC50) and compare them, as shown in Table 5.

TABLE 5
Half-inhibitory concentrations of allopurinol and the synthetic
ploypeptides (IC₅₀)

| | | Molecular | | |
|---|---|---|---|---|
| Sample | Structure | weight | IC₅₀(μg/ml) | IC₅₀(M) |
| Allopurinol | C₅H₄N₄O | 136.11 | 0.26 | 1.91 x 10⁻⁶ |
| S1 | VPSGPLGPEGPR | 1162.2 | 3200 | 2.75 x 10⁻³ |
| S2 | TGPLGPSPGP | 879.2 | 110 | 1.25 x 10⁻⁴ |
| S3 | HDLPLPLPE | 1030.2 | 60 | 5.82 x 10⁻⁵ |
| S4 | DNLPPLPK | 893.5 | 60 | 6.72 x 10⁻⁵ |

The synthetic peptides S1, S2, S3, and S4 reach half-inhibitory concentrations on XOD at 3.2 mg/mL, 110 μg/mL, 60 μg/mL, 60 μg/mL respectively. It is found through experiments that, GSCP2 reach a half-inhibitory concentration on XOD at 5.2 mg/mL under the same reaction system; in addition, allopurinol reach a half-inhibitory concentration on XOD at 0.26 μg/mL, but when the concentration reach 30 μg/mL, XOD still retain about 10% of the enzyme activity, and XOD enzyme activity is completely inhibited when the concentration of S2, S3, or S4 is three times of the half-inhibitory concentration. It is suggested that the artificially synthesized active polypeptides have a different XOD enzyme activity inhibition mechanism from allopurinol.

Analysis 2:

Acute hyperuricemia mouse model: the experimental groups are divided into normal group, model group, positive drug group (allopurinol, with a trademark name of BAILINGWEI, 2 mg/ml), ADCS group (20 mg/ml). GSCP group (40 mg/ml), ten mice per group, each of them is intragastrically administered (20 ml/kg, ultrapure water for normal group and model group) for 7 consecutive days; 1 h after intragastric injection on the seventh day, 0.5 ml of suspension with 15 mg/ml potassium oxazinate (Sangon Biotech Co., Ltd.) and 10 mg/ml hypoxanthine (Sigma-Aldrich LLC.) is injected intraperitoneally for modeling; blood is taken from the liver 1 h after modeling, and the blood uric acid level and the xanthine oxidase activity of liver are detected, the results are shown in FIG. 8.

Hypoxanthine is a precursor of uric acid, and potassium oxazinate is an inhibitor of uricase. After intraperitoneal injection of potassium oxazinate and hypoxanthine, the blood uric acid level in the mice increases significantly, indicating that the mouse model of hyperuricemia is successfully established. Compared with the model group, the blood uric acid level of the mice in the positive drug allopurinol group is close to 0, and the blood uric acid level in the GSCP group is also at a low level, GSCP shows outstanding uric acid lowering activity when it is administered orally. The results of liver XOD enzyme activity test show that, the XOD activity of allopurinol group or GSCP group is lower than that of model group, indicating that GSCP plays a role in lowing uric acid by inhibiting liver XOD activity. The results of the two sets of data show that, the active ingredient playing a role in lowing uric acid in ADCS is not chondroitin sulfate, and it is suggested that the component having uric acid-lowering activity is the polypeptide component in GSCP, i.e., the component of GSCP2.

The foregoing merely illustrates the principles of the present invention, and it should be understood that the scope of the present invention is not intended to be limited to the exemplary aspects described herein, but should include all currently known and future developed equivalents. In addition, it should be pointed out that, without departing from the technical principles of the present invention, several improvements and modifications can be made, and these improvements and modifications should also be regarded as the scope of the present invention.

1. An Andrias davidianus cartilage enzymatic extract, comprising:
an Andrias davidianus chondroitin sulfate, and a crude cartilage peptide extract.

2. The Andrias davidianus cartilage enzymatic extract according to claim 1, wherein the Andrias davidianus chondroitin sulfate contains a non-sulfated disaccharide (CS—O), a disaccharide sulfated at the C6 position (CS—C), and a disaccharide sulfated at the C4 position (CS-A).

3. The Andrias davidianus cartilage enzymatic extract according to claim 1, wherein the Andrias davidianus chondroitin sulfate is composed of a non-sulfated disaccharide (CS—O), a disaccharide sulfated at the C6 position (CS—C), and a disaccharide sulfated at the C4 position (CS-A).

4. The Andrias davidianus cartilage enzymatic extract according to claim 1, wherein the Andrias davidianus chondroitin sulfate has a weight-average molecular weight (Mw) of 40 kDa or more, preferably 45 kDa or more, more preferably 48 kDa or more, most preferably 49.2 kDa.

5. The Andrias davidianus cartilage enzymatic extract according to claim 1, wherein the crude cartilage polypeptide extract comprises one or more of TGPLGPSPGP, HDLPLPLPE, DNLPPLPK, and VPSGPLGPEGPR.

6. The Andrias davidianus cartilage enzymatic extract according to claim 1, wherein the Andrias davidianus chondroitin sulfate accounts for 15% to 30% by mass of the total Andrias davidianus cartilage enzymatic extract, and the crude cartilage polypeptide extract accounts for 70% to 85% by mass of the total Andrias davidianus cartilage enzymatic extract.

7. The Andrias davidianus cartilage enzymatic extract according to claim 1, which is obtained by a method comprising:
obtaining cartilage from Andrias davidianus, proteolyzing the obtained cartilage, and obtaining a proteolyzed supernatant and drying the same.

8-10. (canceled)

11. A cartilage polypeptide, characterized in that the cartilage polypeptide comprises one or more of TGPLGPSPGP, HDLPLPLPE, DNLPPLPK, and VPSGPLGPEGPR.

12. The cartilage polypeptide according to claim 11, which has an activity of inhibiting XOD enzyme.

13. The cartilage polypeptide according to claim 11, which has uric acid-lowering activity.

14. An alcohol precipitation component of the Andrias davidianus cartilage enzymatic extract, comprising an Andrias davidianus chondroitin sulfate, wherein the Andrias davidianus chondroitin sulfate contains a non-sulfated disaccharide (CS—O), a disaccharide sulfated at the C6 position (CS—C), and a disaccharide sulfated at the C4 position (CS-A).

15. The alcohol precipitation component of the Andrias davidianus cartilage enzymatic extract according to claim 14, wherein the Andrias davidianus chondroitin sulfate is composed of a non-sulfated disaccharide (CS—O), a disaccharide sulfated at the C6 position (CS—C), and a disaccharide sulfated at the C4 position (CS-A).

16. The alcohol precipitation component of the Andrias davidianus cartilage enzymatic extract according to claim 14, wherein the Andrias davidianus chondroitin sulfate has a weight-average molecular weight (Mw) of 40 kDa or more, preferably 45 kDa or more, more preferably 48 kDa or more, most preferably 49.2 kDa.

17. The alcohol precipitation component of the Andrias davidianus cartilage enzymatic extract according to claim 14, which is obtained by a method comprising:
obtaining cartilage from Andrias davidianus,
proteolyzing the obtained cartilage,
obtaining a proteolyzed supernatant and drying the same,
alcohol-precipitating the proteolyzed supernatant or the dried matter thereof, and obtaining a precipitate.

18-29. (canceled)

30. The cartilage polypeptide according to claim 11, which is extracted from the alcohol-soluble component of the Andrias davidianus cartilage enzymatic extract.

31. The cartilage polypeptide according to claim 30, wherein the alcohol-soluble component of the Andrias davidianus cartilage enzymatic extract is obtained by a method comprising:
obtaining cartilage from Andrias davidianus,
proteolyzing the obtained cartilage,
obtaining a proteolyzed supernatant and drying the same,
alcohol-precipitating the proteolyzed supernatant or the dried matter thereof, and
obtaining a supernatant.

32. The Andrias davidianus cartilage enzymatic extract according to claim 7, wherein the protease is an alkaline protease, preferably 2709 protease, and the proteolysis is performed under the conditions of pH 9-11.

33. The cartilage polypeptide according to claim 31, wherein the protease is an alkaline protease, preferably 2709 protease, and the proteolysis is performed under the conditions of pH 9-11.

34. The alcohol precipitation component of the Andrias davidianus cartilage enzymatic extract according to claim 17, wherein the protease is an alkaline protease, preferably 2709 protease, and the proteolysis is performed under the conditions of pH 9-11.

35. The alcohol precipitation component of the Andrias davidianus cartilage enzymatic extract according to claim 17, wherein the mass concentration of ethanol in the alcohol precipitation operation is 50%-90%, preferably is 70%.
